(19) United States
(12) Patent Application Publication
(10) Pub. No.: US 2026/0094684 A1
(43) Pub. Date: Apr. 2, 2026

(54) PUPILLARY BIOMARKERS TO ASSESS EFFECTS OF PSYCHOTROPIC DRUGS

(71) Applicant: Neuroptics, Inc., Irvine, CA (US)

(72) Inventors: Kamran Siminou, Newport Coast, CA (US); Claudio Privitera, Albany, CA (US); Jeffrey Oliver, Cedar Park, TX (US)

(21) Appl. No.: 18/898,899

(22) Filed: Sep. 27, 2024

Related U.S. Application Data
(60) Provisional application No. 63/536,097, filed on Sep. 1, 2023.

(51) Int. Cl. G16H 20/10; A61B 3/11; A61B 3/14

(52) U.S. Cl. CPC G16H 20/10; A61B 3/112; A61B 3/14

(57) ABSTRACT

A method of assessing the effectiveness of a psychotropic drug on a mammalian subject is described. The method includes providing a pupilometer having a display, using the pupilometer to obtain pupillary biomarker data from at least one pupil of the subject before the subject is administered the psychotropic drug, using the pupilometer to obtain pupillary biomarker data from at least one pupil of the subject after the subject is administered the psychotropic drug, using the pupilometer to process all of the pupillary biomarker data, and displaying an output on the display of the pupilometer that indicates the effectiveness of the psychotropic drug.

### CROSS REFERENCE TO RELATED APPLICATIONS

This application claims the benefit of priority to U.S. Ser. No. 63/536,097, filed Sep. 1, 2023 entitled “Pupillary Biomarkers To Assess Effects Of Psychotropic Drugs”.

### BACKGROUND

The present disclosure relates to methods, systems, programs and devices for assessing the effects and/or effectiveness of psychotropic drugs. The treatment of psychological disorders such as depression, anxiety, and OCD, with drugs such as SSRI'is at an all-time high. The majority of patients are prescribed three or more trials of medication, in a hit-or-miss fashion, until an effective medication at an effective dose is determined. The determination of effectiveness of these medications is often subjective, relying on self-reporting of symptoms from the patient. Typically, each trial of medication takes six weeks or more to determine effectiveness and to tune dosing once identified. This protracted approach to treatment protocols results in extended suffering with frequent negative consequences such as exacerbated symptoms of depression, with lethargy and a sense of hopelessness often reported, even when a drug may actually be starting to work. Direct objective biomarkers that can be detected and assessed earlier in the course of treatment than traditional extended trial of drug regiments are needed. In addition, biomarkers for psychotropic drug effectiveness that remove subjectivity from the assessment are also needed. Therefore, a biomarker which can objectively measure psychotropic drug effectiveness at an early stage of treating mental disorders would be extremely beneficial at improving care, by enabling a clinician to make informed decisions with expediency compared to current treatment protocols.

### SUMMARY OF THE DISCLOSURE

Pupillometers offer the potential of such objective measures of drug effectiveness due to the integration of autonomic input to the midbrain. The midbrain, which mediates pupillary response to stimuli via the hypothalamus and midbrain (EWN) can therefore be a proxy to overall autonomic tone. This sympathetic tone and changes induced by medications may be probed with techniques such as the pupil light reflex, pupil dilation to pain or other noxious stimulus and also by monitoring fluctuations in the baseline size (noise) in the absence of other stimuli.

In one embodiment, a method of assessing the effectiveness of a psychotropic drug on a mammalian subject is described. The method includes providing a pupilometer having a display, using the pupilometer to obtain pupillary biomarker data from at least one pupil of the subject before the subject is administered the psychotropic drug, using the pupilometer to obtain pupillary biomarker data from at least one pupil of the subject after the subject is administered the psychotropic drug, using the pupilometer to process all of the pupillary biomarker data, and displaying an output on the display of the pupilometer that indicates the effectiveness of the psychotropic drug.

In another embodiment, a method of assessing the effectiveness of a psychotropic drug on a mammalian subject includes providing a pupilometer having a display, said pupilometer having access to a dataset of pupillary biomarker data obtained from subjects other than the subject, using the pupilometer to obtain pupillary biomarker data from at least one pupil of the subject, using the pupilometer to process all of the pupillary biomarker data, and displaying an output on the display of the pupilometer that indicates the effectiveness of the psychotropic drug.

In another embodiment, a program product is described. The program product includes a computer-readable medium and computer-executable instructions recorded on the computer-readable medium for performing a method of assessing the effectiveness of a psychotropic drug on a mammalian subject, wherein the method includes: causing a pupilometer having a display to obtain pupillary biomarker data from at least one pupil of the subject before the subject is administered the psychotropic drug; causing the pupilometer to obtain pupillary biomarker data from at least one pupil of the subject after the subject is administered the psychotropic drug; causing the pupilometer to process all of the pupillary biomarker data; and causing the pupilometer to display on its display an output that indicates the effectiveness of the psychotropic drug.

In another embodiment, a program product is described. The program product includes a computer-readable medium and computer-executable instructions recorded on the computer-readable medium for performing a method of assessing the effectiveness of a psychotropic drug on a mammalian subject, wherein the method includes: causing a pupilometer to access a dataset of pupillary biomarker data obtained from subjects other than the subject; causing the pupilometer to obtain pupillary biomarker data from at least one pupil of the subject; causing the pupilometer to process all of the pupillary biomarker data; causing the pupilometer to display on its display an output that indicates the effectiveness of the psychotropic drug.

In yet another embodiment, a pupilometer is disclosed. The pupilometer has a light source for generating and emitting a light stimulus to stimulate a pupil of a mammalian subject; a camera for recording and measuring a dynamic response of said pupil to the light stimulus; a memory that can store data relating to the dynamic response of the pupil to the light stimulus; a microprocessor that can process data obtained from a pupil prior to the subject being administered a psychotropic drug and data obtained from a pupil after the subject is administered a psychotropic drug; and a display for displaying an output indicative of the effectiveness of the psychotropic drug.

Other features and advantages will be apparent from the following description of the various embodiments of the disclosure, which illustrate, by way of example, the principles of the disclosed devices and methods.

### DETAILED DESCRIPTION

A pupillometer, either handheld or desktop mounted, is configured to collect pupil information. This information may be a pupil light reflex, a pupil dilation reflex to a light, noxious or pleasurable stimulus (hereinafter all forms of stimulus are referred to as “stimulus”) or a measure of baseline pupillary fluctuations known as pupillary noise or any combination of these pupil measurements. A sequence of measurements is collected on a patient at different time points before and after drug therapy is commenced. The measurements may be compared from time point-to-time-point (intrasubject) or against a database of previously known (inter-subject) pupil parameters which are indicative of autonomic tone or changes in autonomic tone due to medications. A recommendation of initial medication can be made based on measured pupil parameters. An effectiveness scale will be reported for the medications given to a patient based on measured pupil parameters. A determination of compliance to medication protocols, assuring a patient is taking their drugs, may be made based on pupil measurements collected over the course of several months or years.

The pupillometer has features that enable it to monitor in real-time the response of an individual's pupils to a stimulus, such as a light stimulus. In the examples described herein the pupillometer has a light stimulus source built into it. Variations on the type of stimulus and the arrangement of that stimulus in relation to the pupillometer, such as those variations described in U.S. Pat. No. 7,147,327, are also contemplated and incorporated herein by reference. The working components, electronics, and software, other than those described below, are also fully described in, e.g., U.S. Pat. No. 7,147,327, and U.S. patent application Ser. No. 17/496,109, both of which are incorporated herein by reference in their entirety, and will therefore not be further discussed herein. As used in this disclosure, pupillometer also means a smart phone or other hand-held electronic device (or computer) that runs an application or software that imparts pupillometry capabilities, such as identifying a pupil and recording and measuring its dynamic response to a stimulus, such as a light stimulus.

While the invention is susceptible to various modifications and alternative forms, specific examples thereof have been shown by way of example in the drawings and are herein described in detail. It should be understood, however, that the invention is not to be limited to the particular forms or methods disclosed, but to the contrary, the invention is to cover all modifications, equivalents, and alternatives falling within the spirit and scope of the appended claims.

We claim:

1. A method of assessing the effectiveness of a psychotropic drug on a mammalian subject, said method comprising:
providing a pupilometer;
using the pupilometer to obtain pupillary biomarker data from at least one pupil of the subject before the subject is administered the psychotropic drug;
using the pupilometer to obtain pupillary biomarker data from at least one pupil of the subject after the subject is administered the psychotropic drug;
using the pupilometer to process all of the pupillary biomarker data;
displaying an output that indicates the effectiveness of the psychotropic drug.

2. A method of assessing the effectiveness of a psychotropic drug on a mammalian subject, said method comprising:
providing a pupilometer having a display, said pupilometer having access to a dataset of pupillary biomarker data obtained from subjects other than the subject
using the pupilometer to obtain pupillary biomarker data from at least one pupil of the subject;
using the pupilometer to process all of the pupillary biomarker data;
displaying an output on the display of the pupilometer that indicates the effectiveness of the psychotropic drug.

3. A program product comprising a computer-readable medium and computer-executable instructions recorded on the computer-readable medium for performing a method of assessing the effectiveness of a psychotropic drug on a mammalian subject, wherein the method comprises:
causing a pupilometer having a display to obtain pupillary biomarker data from at least one pupil of the subject before the subject is administered the psychotropic drug;
causing the pupilometer to obtain pupillary biomarker data from at least one pupil of the subject after the subject is administered the psychotropic drug;
causing the pupilometer to process all of the pupillary biomarker data;
causing the pupilometer to display on its display an output that indicates the effectiveness of the psychotropic drug.

4. A program product comprising a computer-readable medium and computer-executable instructions recorded on the computer-readable medium for performing a method of assessing the effectiveness of a psychotropic drug on a mammalian subject, wherein the method comprises:
causing a pupilometer to access a dataset of pupillary biomarker data obtained from subjects other than the subject;
causing the pupilometer to obtain pupillary biomarker data from at least one pupil of the subject;
causing the pupilometer to process all of the pupillary biomarker data;
causing the pupilometer to display on its display an output that indicates the effectiveness of the psychotropic drug.

5. A pupilometer comprising:
a light source for generating and emitting a light stimulus to stimulate a pupil of a mammalian subject;
a camera for recording and measuring a dynamic response of said pupil to the light stimulus;
a memory that can store data relating to the dynamic response of the pupil to the light stimulus;
a microprocessor that can process data obtained from a pupil prior to the subject being administered a psychotropic drug and data obtained from a pupil after the subject is administered a psychotropic drug and provide an output indicative of the effectiveness of the psychotropic drug.

6. The pupilometer of claim 5, further comprising a display for displaying the output indicative of the effectiveness of the psychotropic drug.
